(19) United States
(12) Patent Application Publication
(10) Pub. No.: US 2024/0052317 A1
(43) Pub. Date: Feb. 15, 2024

(54) Methods for Differentiating Stromal Cells or Pericytes

(71) Applicant: Lung Biotechnology PBC, Silver Spring, MD (US)

(72) Inventors: Jason PARISE, Silver Spring, MD (US); Kurt WONG, Silver Spring, MD (US)

(21) Appl. No.: 18/354,205

(22) Filed: Jul. 18, 2023

Related U.S. Application Data
(60) Provisional application No. 63/390,458, filed on Jul. 19, 2022.

(51) Int. Cl. C12N 5/077; C12N 5/00

(52) U.S. Cl. CPC C12N 5/0669; C12N 5/0018; C12N 2501/155; C12N 2501/165; C12N 2501/16; C12N 2501/999; C12N 2501/15; C12N 2501/135; C12N 2513/00

(57) ABSTRACT

The present disclosure relates to methods of differentiating pluripotent stem cells into stromal cells or pericytes. The present disclosure also relates to stromal cells or pericytes made by such methods, organoids containing such stromal cells or pericytes, and methods of using the same. The present disclosure also relates to differentiation media for use in the same.

### CROSS REFERENCE TO RELATED APPLICATIONS

This application claims the priority benefit of U.S. Provisional Application No. 63/390,458, filed Jul. 19, 2022, which is incorporated herein by reference in its entirety.

### FIELD OF THE DISCLOSURE

The present disclosure relates to methods of differentiating pluripotent stem cells into stromal cells or pericytes. The present disclosure also relates to stromal cells or pericytes made by such methods, organoids containing such stromal cells or pericytes, and methods of using the same. The present disclosure also relates to differentiation media for use in the same.

### BACKGROUND

Pluripotent stem cells (PSC) are undifferentiated or partially differentiated cells that can differentiate into various other cell types. Induced pluripotent stem cells (iPSC) are a type of PSC derived from adult somatic cells that have been genetically reprogrammed to an embryonic stem cell (ESC)-like state through the expression of genes and factors important for maintaining the defining properties of ESC. iPSC have generated interest in the medical community recently because they address many obstacles associated with the use of embryonic stem cells, and allow for the generation of patient-specific PSC, which can be genetically corrected, differentiated into adult lineages, and returned to the same patient as an autograft. Yamanaka et al., Cell Stem Cell. 1(1):39-49 (2007); Nishikawa et al., Nat. Rev. Mol. Cell Biol. 9:725 (2008). In addition to genetic disorders, iPSC can be used for tissue regeneration and disease modeling. Kogut et al., Methods Mol. Biol. 1195:1-12 (2014). PSC and iPSC can be differentiated into many different cell types, including endothelial cells (EC). Jang et al., Am. J. Pathol. 189(3):502-512 (2019); Gu et al., Curr. Protoc. Hum. Genet. published online 2018 Jul. 6. doi: 10.1002/cphg.64.

### BRIEF SUMMARY

The present disclosure provides methods for differentiating pluripotent stem cells (PSC) into stromal cells or pericytes.

In some aspects, the method comprises (i) culturing PSC (e.g., iPSC) in a base culture medium comprising bone morphogenetic protein 4 (BMP4), vascular endothelial growth factor (VEGF), a glycogen synthase kinase 3 (GSK3) inhibitor, activin A, and a Rho-associated, coiled-coil containing protein kinase (ROCK) inhibitor; (ii) culturing the cells of (i) in a base culture medium comprising VEGF and a transforming growth factor β (TGFβ) inhibitor; and (iii) culturing the cells of (ii) in a base culture medium comprising TGFβ3 and a platelet-derived growth factor (PDGF) to form stromal cells or pericytes.

In some aspects, the method further comprises culturing the cells of (ii) in a base culture medium before (iii).

In some aspects, the method further comprises (iv) culturing the cells of (iii) in a base culture medium.

In some aspects, the cells of (ii) are not passaged before (iii).

In some aspects, the method comprises (i) culturing PSC (e.g., iPSC) in a base culture medium comprising BMP4, VEGF, a GSK3 inhibitor, and activin A; (ii) culturing the cells of (i) in a base culture medium comprising VEGF and a TGFβ inhibitor; (iii) culturing the cells of (ii) in a base culture medium; and (iv) culturing the cells of (iii) in a base culture medium comprising TGFβ3 and a PDGF to form stromal cells.

In some aspects, the method further comprises (v) culturing the cells of (iv) in a base culture medium.

In some aspects, the cells of (ii) are not passaged before (iii).

In some aspects, the culture medium of (i) further comprises a ROCK inhibitor. In some aspects, the ROCK inhibitor is Y-27632. In some aspects, Y-27632 is present in the culture medium at a concentration of about 10 μM.

In some aspects, the GSK3 inhibitor is CHIR99021. In some aspects, CHIR99021 is present in the culture medium at a concentration of about 1.5 μM.

In some aspects, BMP4 is present in the culture medium at a concentration of about 30 ng/mL.

In some aspects, VEGF is present in the culture medium at a concentration of about 50 ng/mL.

In some aspects, activin A is present in the culture medium at a concentration of about 25 ng/mL.

In some aspects, the TGFβ inhibitor is SB431542. In some aspects, SB431542 is present in the culture medium at a concentration of about 10 μM.

In some aspects, TGFβ3 is present in the culture medium at a concentration of about 2 ng/mL.

In some aspects, the PDGF is PDGF-BB. In some aspects, PDGF-BB is present in the culture medium at a concentration of about 4 ng/mL.

In some aspects, the stromal cells are pericytes.

In some aspects, the iPSC are not fully confluent at the start of the culturing of (i).

In some aspects, the iPSC are at from about 50% to about 80% confluency at the start of the culturing of (i).

The present disclosure also provides stromal cells made by a differentiation method disclosed herein, pericytes made by a differentiation method disclosed herein, an organoid containing the stromal cells disclosed herein, an organoid containing pericytes disclosed herein, and certain methods of use thereof.

The present disclosure also provides certain differentiation media. In some aspects, the differentiation medium comprises a base culture medium, BMP4, VEGF, a GSK3 inhibitor, activin A, and a ROCK inhibitor. In some aspects, the differentiation medium comprises a base culture medium, about 30 ng/mL BMP4, about 50 ng/mL VEGF, about 1.5 μM CHIR99021, about 25 ng/mL activin A, and about 10 μM Y-27632.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1A is an exemplary whole-cell image from day 1 of the differentiation protocol described in Example 1.

FIG. 1B is an exemplary whole-cell image from day 2 of the differentiation protocol described in Example 1.

FIG. 1C is an exemplary whole-cell image from day 4 of the differentiation protocol described in Example 1.

FIG. 1D is an exemplary whole-cell image from day 5 of the differentiation protocol described in Example 1.

FIG. 1E is an exemplary whole-cell image from day 7 of the differentiation protocol described in Example 1.

FIG. 1F is an exemplary whole-cell image from day 9 of the differentiation protocol described in Example 1.

FIG. 1G is an exemplary whole-cell image from day 10 of the differentiation protocol described in Example 1.

FIG. 1H is an exemplary whole-cell image from day 17 of the differentiation protocol described in Example 1

FIG. 1I-1J are exemplary whole-cell images of final test differentiated cells described in Example 1.

FIG. 2 shows tube formation results from test differentiated cells of Example 1 co-cultured with human pulmonary artery endothelial cells (HPAEC) treated with or without the angiogenesis inhibitor, imatinib mesylate.

FIG. 3A-3C shows exemplary images from the three-hour timepoint for tube formation of co-cultures of HPAEC and test differentiated cells of Example 1 (FIG. 3A), HPAEC and test differentiated cells treated with imatinib mesylate (FIG. 3B), and HPAEC and placental pericytes (FIG. 3C).

### DETAILED DESCRIPTION

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### II. Differentiation Methods

The present disclosure relates to improved methods of differentiating pluripotent stem cells (PSC) into stromal cells or pericytes. Such methods result, for example, in improved cell viability, yield and/or characteristics of the differentiated cells.

As used herein, the terms “differentiation” and “differentiating” refer to the process of inducing or reprogramming young or immature cells (e.g., pluripotent stem cells) into more mature or specialized cells (e.g., stromal cells or pericytes). In general, differentiation of pluripotent stem cells can be effected, for example, by changing culturing conditions of the cells, such as changing the stimuli agents in a culture medium or the physical state of the cells.

As used herein, the terms “pluripotent stem cell,” “pluripotent stem cells,” and “PSC” refer to young or immature cell(s) that can develop into more mature or specialized cells (e.g., stromal cells or pericytes).

In some aspects, PSC include, but are not limited to, embryonic stem cells (ESC), induced pluripotent stem cells (iPSC), embryonic germ cells, adult stem cells, or a combination thereof. In some aspects, the PSC are from a human. In some aspects, the PSC are from an animal. In some aspects, the animal is a sheep, pig or primate.

As used herein, the terms “induced pluripotent stem cell,” “induced pluripotent stem cells,” and “iPSC” refer to cells produced from differentiated adult, neonatal or fetal cells that have been induced or reprogrammed into pluripotent stem cells.

As used herein, the terms “stromal cells,” “stromal cell,” or “SC” refer to cells that function as connective tissue cells of an organ which support the function of the parenchymal cells of the particular organ. As used herein, a stromal cell includes a mature stromal cell, a stromal progenitor cell, and a stromal precursor cell.

In some aspects, a stromal cell described herein is a pericyte. As used herein, the terms “pericyte” and “pericytes” refer to fibroblast-like cells with extensive cytoplasmic processes that wrap around endothelial cells in arterioles, capillaries, and venules, covering between 22% and 99% of the endothelial cell surface. Pericytes are embedded in the basement membrane of blood capillaries, where they communicate with endothelial cells by means of both direct physical contact and paracrine signaling.

In some aspects, the differentiation methods provided herein include certain cell culturing conditions, such as culturing cells in certain culture media.

As used herein, the terms “cell culture,” “cell culturing,” “culture,” “culturing,” and “cultured” refer to the maintenance, growth and/or differentiation of cells in an in vitro environment. The terms “cell culture medium,” “cell culture media,” “culture medium” and “culture media” refer to a composition for culturing cells that contains nutrients to maintain cell viability, support proliferation and optionally differentiation. A cell culture medium can contain one or more of the following: salt(s), buffer(s), amino acid(s), glucose or other sugar(s), antibiotic(s), serum or serum replacement, and other components such as growth factors, vitamins, etc.

In some aspects, the differentiation methods provided herein refer to a cell culture media as a “base culture medium” supplemented with other components (sometimes referred to herein as “differentiation media” or “differentiation medium”). As used herein, a “base culture medium” or “base culture media” refer to a composition that contains the minimal elements required for maintenance, growth and/or differentiation of cells in an in vitro environment. Examples of a base culture medium include, but are not limited to, Dulbecco's Modified Eagle Medium (DMEM), MEM, Iscove's Modified Dulbecco's Medium (IMDM), Glasgow's modified MEM (GMEM), DMEM/F12, Leibovitz L-15, RPMI-1640, CMRL, Ham F10, and Ham F12. In some aspects, a base culture medium is supplemented with one or more other components such as amino acid(s), antibiotic(s), serum, growth factor(s), etc. Such components are well known in the art and described further herein.

In some aspects, the differentiation methods provided herein include certain cell culturing conditions, such passaging cells in certain culture media. As used herein, the terms “passage,” “passaged,” and “passaging” refer to the act of subdividing and plating cells at a lower concentration into one or more cell culture surfaces or vessels, when the cells have proliferated to a desired extent. Passaging typically involves detaching cells by mechanical or enzymatic methods (e.g., incubation in Accutane®) before plating, optionally at a certain cell density. Methods for passaging cells are well known and described further herein.

In some aspects, culturing and passaging in the differentiation methods provided herein are performed with one or more substrates coated onto the cell culture surface or vessel. Such substrates include, but are not limited to, vitronectin, gelatin, laminin, fibronectin, collagen (e.g., collagen I, collagen IV, or a combination thereof), elastin, osteopontin, thrombospondin, mixtures of naturally occurring cell line-produced matrices such as Matrigel™, and synthetic or man-made surfaces such as polyamine monolayers and carboxy-terminated monolayers, or a combination thereof. Methods for coating a substrate onto a cell culture surface or vessel are well known and described further herein.

Some aspects of the differentiation methods provided herein include culturing cells in a base culture medium containing bone morphogenetic protein 4 (BMP4). BMP4 stimulates differentiation of overlying ectodermal tissue and is known to stimulate bone formation in adult animals.

In some aspects, BMP4 is present in the base culture medium at a concentration of from about 10 ng/mL to about 50 ng/mL, or any value or range of values thereof, including, for example, from about 20 ng/mL to about 50 ng/mL, from about 30 ng/mL to about 50 ng/mL, from about 40 ng/mL to about 50 ng/mL, from about 10 ng/mL to about 40 ng/mL, from about 20 ng/mL to about 40 ng/mL, from about 30 ng/mL to about 40 ng/mL, from about 10 ng/mL to about 30 ng/mL, from about 20 ng/mL to about 30 ng/mL, or from about 10 ng/mL to about 20 ng/mL. In some aspects, BMP4 is present in the base culture medium at a concentration of about 10 ng/mL, about 20 ng/mL, about 30 ng/mL, about 40 ng/mL or about 50 ng/mL. In some aspects, BMP4 is present in the base culture medium at a concentration of about 30 ng/mL.

In other aspects, a base culture medium of the differentiation methods provided herein does not comprise or is essentially free of BMP4. As used herein, the term “essentially free” refers to a culture medium that is at least 95% free, 96% free, 97% free, 98% free, 99% free or 100% free of BMP4 or has an undetectable amount of BMP4, as measured by methods known in the art and described further herein. The terms “do not comprise” and “does not comprise” refer to a culture medium that does not contain BMP4, or has an undetectable amount of BMP4, as measured by methods known in the art and described further herein.

Some aspects of the differentiation methods provided herein include culturing cells in a base culture medium containing vascular endothelial growth factor (VEGF). VEGF is a signaling protein that promotes the growth of new blood vessels. VEGF forms part of the mechanism that restores the blood supply to cells and tissues when they are deprived of oxygenated blood due to compromised blood circulation.

In some aspects, VEGF is present in the base culture medium at a concentration of from about 10 ng/mL to about 100 ng/mL, or any value or range of values thereof, including, for example, from about 25 ng/mL to about 100 ng/mL, from about 50 ng/mL to about 100 ng/mL, from about 75 ng/mL to about 100 ng/mL, from about 10 ng/mL to about 75 ng/mL, from about 25 ng/mL to about ng/mL, from about 50 ng/mL to about 75 ng/mL, from about 10 ng/mL to about 50 ng/mL, from about 25 ng/mL to about 50 ng/mL, or from about 10 ng/mL to about 25 ng/mL. In some aspects, VEGF is present in the base culture medium at a concentration of about 10 ng/mL, about 25 ng/mL, about 50 ng/mL, about 75 ng/mL or about 100 ng/mL. In some aspects, VEGF is present in the base culture medium at a concentration of about 50 ng/mL.

Some aspects of the differentiation methods provided herein include culturing cells in a base culture medium containing a glycogen synthase kinase 3 (GSK3) inhibitor. GSK3 is a serine/threonine protein kinase that mediates the addition of phosphate molecules on certain serine and threonine amino acids of cellular substrates (e.g., glycogen synthase). This phosphorylation typically results in inhibition of the substrates. GSK3 has also been implicated in the control of cellular response to damaged DNA and Wnt signaling and phosphorylation of Ci in the Hedgehog (Hh) pathway, targeting it for proteolysis to an inactive form.

As used herein, a “GSK3 inhibitor” refers to a compound that inhibits one or more GSK3 enzymes. The family of GSK3 enzymes is well known and a number of variants have been described (e.g., Schaffer et al., Gene, 302:73-81, 2003). Specific examples of GSK3 inhibitors include, but are not limited to, Kenpaullone, 1-Azakenpaullone, CHIR99021, CHIR98014, AR-A014418, CT99021, CT20026, SB415286, SB216763, AR-A014418, lithium, SB 415286, and TDZD-8. Further exemplary GSK3 inhibitors include, but are not limited to, BIO (2′Z,3£)-6-Bromomdirubm-3′-oxime (GSK3 Inhibitor IX); BIO-Acetoxime (2′Z,3′E)-6-Bromoindirubin-3′-acetoxime (GSK3 Inhibitor X); (5-Methyl-1H-pyrazol-3-yl)-(2-phenylquinazolin-4-yl)amine (GSK3 Inhibitor XIII); Pyridocarbazole-cyclopenadienylruthenium complex (GSK3 Inhibitor XV); TDZD-8,4-Benzyl-2-methyl-1,2,4-thiadiazolidine-3,5-dione (GSK3beta Inhibitor I); 2-Thio(3-iodobenzyl)-5-(1-pyridyl)-[1,3,4]-oxadiazole (GSK3beta Inhibitor II); OTDZT 2,4-Dibenzyl-5-oxothiadiazolidine-3-thione (GSK3beta Inhibitor III); alpha-4-Dibromoacetophenone (GSK3beta Inhibitor VII); AR-AO 14418 N-(4-Methoxybenzyl)-N′-(5-nitro-1,3-thiazol-2-yl)urea (GSK-3beta Inhibitor VIII); 3-(1-(3-Hydroxypropyl)-1H-pyrrolo[2,3-b]pyridin-3-yl]-4-pyrazin-2-yl-pyrrole-2,5-dione (GSK3beta Inhibitor XI); TWS1 19-pyrrolopyrimidine compound (GSK3beta Inhibitor XII); L803 H-KEAPP APPQSpP-NH2 or its Myristoylated form (GSK3beta Inhibitor XIII); 2-Chloro-1-(4,5-dibromo-thiophen-2-yl)-ethanone (GSK3beta Inhibitor VI); AR-AO 144-18; SB216763; and SB415286. In some aspects, the GSK3 inhibitor is CHIR99021.

In some aspects, the GSK3 inhibitor (e.g., CHIR99021) is present in the base culture medium at a concentration of from about 0.5 μM to about 3 μM, or any value or range of values thereof, including, for example, from about 0.5 μM to about 1.5 μM, from about 0.5 μM to about 1 μM, from about 0.5 μM to about 1.5 μM, from about 0.5 μM to about 1 μM, or from about 0.5 μM to about 1 μM. In some aspects, the GSK3 inhibitor (e.g., CHIR99021) is about 0.5 μM, about 1 μM, about 1.5 μM or about 3 μM. In some aspects, the GSK3 inhibitor (e.g., CHIR99021) is about 1.5 μM.

Some aspects of the differentiation methods provided herein include culturing cells in a base culture medium containing activin A. Activin A is a dimeric glycoprotein, which belongs to the transforming growth factor β (TGFβ) family. It regulates several biological functions, including hormonal homoeostasis, gonadal functions, muscle growth, immunity, inflammation, and bone remodeling.

In some aspects, activin A is present in the base culture medium at a concentration of from about 5 ng/mL to about 50 ng/mL, or any value or range of values thereof, including, for example, from about 10 ng/mL to about 50 ng/mL, from about 25 ng/mL to about 50 ng/mL, from about 5 ng/mL to about 25 ng/mL, from about 10 ng/mL to about 25 ng/mL or from about 5 ng/mL to about 10 ng/mL. In some aspects, activin A is present in the base culture medium at a concentration of about 5 ng/mL, about 10 ng/mL, about 25 ng/mL or about 50 ng/mL. In some aspects, activin A is present in the base culture medium at a concentration of about 25 ng/mL.

Some aspects of the differentiation methods provided herein include culturing cells in a base culture medium containing a Rho associated kinase (ROCK) inhibitor. ROCK is a serine/threonine kinase that serves downstream effectors of Rho kinases, of which three isoforms exist (RhoA, RhoB and RhoC). A “ROCK inhibitor” can, for example, decrease ROCK expression and/or ROCK activity. Examples of a ROCK inhibitor include, but are not limited to, polynucleotides, polypeptides, and small molecules. More specific examples of a ROCK inhibitor include, but are not limited to, an anti-ROCK antibody, and dominant-negative ROCK variant, siRNA, shRNA, miRNA and antisense nucleic acids that target ROCK. Other examples of a ROCK inhibitor include, but are not limited to, thiazovivin, Y-27632, Fasudil, AR122-86, Y-30141, WF-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some aspects, the ROCK inhibitor is Y-27632.

In some aspects, the ROCK inhibitor (e.g., Y-27632) is present in the base culture medium at a concentration of from about 1 μM to about 20 μM, or any value or range of values thereof, including, for example, from about 1 μM to about 15 μM, from about 1 μM to about 10 μM, from about 1 μM to about 5 μM, from about 5 μM to about 20 μM, from about 5 μM to about 15 μM, from about 5 μM to about 10 μM, from about 10 μM to about 20 μM, from about 10 μM to about 15 μM, or from about 15 μM to about 20 μM. In some aspects, the ROCK inhibitor (e.g., Y-27632) is present in the base culture medium at a concentration of about 1 μM, about 5 μM, about 10 μM, about 15 μM, or about 20 μM. In some aspects the ROCK inhibitor (e.g., Y-27632) is present in the base culture medium at a concentration of about 10 μM.

Some aspects of the differentiation methods provided herein include culturing cells in a base culture medium containing a TGFβ inhibitor. TGFβ is a highly pleiotropic cytokine that plays an important role in wound healing, angiogenesis, immunoregulation and cancer. As used herein, the terms “TGFβ inhibitors” and “TGFβ inhibitor” include, but are not limited to, any inhibitors of TGF signaling in general or inhibitors specific for TGFβ receptor (e.g., ALK5) inhibitors, which can include antibodies to, dominant-negative variants of, and siRNA and antisense nucleic acids that suppress expression of TGFβ receptors. Examples of TGFβ inhibitors include, but are not limited to, SB431542, A-83-01 (also known as 3-(6-Methyl-2-pyridinyl)-N-phenyl-4-(4-quinolinyl)-1H-pyrazole-1-carbothioamide), 2-(3-(6-Methylpyridin-2-yl)-1H-pyrazol-4-yl)-1, 5-naphthyridine, Wnt3a/BIO, BMP4, GW788388 (−4-[3-(pyridin-2-yl)-1H-pyrazol-4-yl]pyridm-2-yl}-N-(tetrahydro-2H-pyran-4-yl)benzamide), SMI 6, 3-((5-(6-methylpyridin-2-yl)-4-(quinoxalin-6-yl)-1H-imidazol-2-yl)methyl)benzami, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), SU5416, lerdelimumb (CAT-152); metelimumab (CAT-192); GC-1008; IDI 1; AP-12009; AP-1 1014; LY550410; LY580276; LY364947; LY2109761; SB-431542; SD-208; SM16; NPC-30345; KÏ26894; SB-203580; SD-093; ALX-270-448; EW-7195; SB-525334; FN-1233; SKI2162; Gleevec; 3,5,7,2′,4′-pentahydroxyfiavone (Morin); activin-M108A; P144; soluble TBR2-Fc; and pyrimidine derivatives and indolinones reported in Roth et al., 2010. In some aspects, the TGFβ inhibitor is SB431542.

In some aspects, the TGFβ inhibitor (e.g., SB431542) is present in the base culture medium at a concentration of from about 1 μM to about 20 μM, or any value or range of values thereof, including, for example, from about 5 μM to about 20 μM, from about 10 μM to about 20 μM, from about 1 μM to about 10 μM, and from about 1 μM to about 5 μM. In some aspects, the TGFβ inhibitor (e.g., SB431542) is present in the base culture medium at a concentration of about 1 μM, about 5 μM, about 10 μM, or about 20 μM. In some aspects, the TGFβ inhibitor (e.g., SB431542) is present in the base culture medium at a concentration of about 10 μM.

Some aspects of the differentiation methods provided herein include culturing cells in a base culture medium containing transforming growth factor β3 (TGFβ3). TGFβ3 is a cytokine that is involved in cell differentiation, embryogenesis and development. TGFβ3 is believed to regulate molecules involved in cellular adhesion and extracellular matrix (ECM) formation during the process of palate development.

In some aspects, TGFβ3 is present in the base culture medium at a concentration of from about 0.5 ng/mL to about 5 ng/mL, or any value or range of values thereof, including, for example, from about 1 ng/mL to about 5 ng/mL, from about 2 ng/mL to about 5 ng/mL, from about 0.5 ng/mL to about 2 ng/mL, from about 1 ng/mL to about 2 ng/mL or from about 0.5 ng/mL to about 1 ng/mL. In some aspects, TGFβ3 is present in the base culture medium at a concentration of about 0.5 ng/mL, about 1 ng/mL, about 2 ng/mL or about 5 ng/mL. In some aspects, TGFβ3 is present in the base culture medium at a concentration of about 2 ng/mL.

Some aspects of the differentiation methods provided herein include culturing cells in a base culture medium containing a platelet-derived growth factor (PDGF). PDGF is a growth factor that regulates cell growth and division, playing a significant role in blood vessel formation, growth of blood vessels from already-existing blood vessel tissue, mitogenesis, and chemotaxis of mesenchymal cells. PDGF is a dimeric glycoprotein that can be composed of two A subunits (PDGF-AA), two B subunits (PDGF-BB), or one of each (PDGF-AB). In some aspects, the PDGF is PDGF-BB.

In some aspects, the PDGF (e.g., PDGF-BB) is present in the base culture medium at a concentration of from about 2 ng/mL to about 8 ng/mL, or any value or range of values thereof, including, for example, from 4 ng/mL to about 8 ng/mL, from about 5 ng/mL to about 8 ng/mL, from about 2 ng/mL to about 5 ng/mL, from about 4 ng/mL to about 5 ng/mL, or from about 2 ng/mL to about 4 ng/mL. In some aspects, the PDGF (e.g., PDGF-BB) is about 2 ng/mL, about 4 ng/mL, about 5 ng/mL, or about 8 ng/mL. In some aspects, the PDGF (e.g., PDGF-BB) is about 4 ng/mL.

In some aspects, a differentiation method provided herein comprises culturing PSC (e.g., iPSC) in a base culture medium comprising BMP4, VEGF, a GSK3 inhibitor (e.g., CHIR99021), activin A, and a ROCK inhibitor (e.g., Y-27632). In some aspects, the base culture medium comprises from about 10 ng/mL to about 50 ng/mL BMP4, from about 10 ng/mL to about 100 ng/mL VEGF, from about 0.5 μM to about 3 μM of a GSK3 inhibitor (e.g., CHIR99021), from about 5 ng/mL to about 50 ng/mL activin A, and from about 1 μM to about 20 μM of a ROCK inhibitor (e.g., Y-27632). In some aspects, the base culture medium comprises about 30 ng/mL BMP4, about 50 ng/mL VEGF, about 1.5 μM of a GSK3 inhibitor (e.g., CHIR99021), about 25 ng/mL activin A, and about 10 μM of a ROCK inhibitor (e.g., Y-27632).

In some aspects, a differentiation method provided herein comprises culturing cells in a base culture medium comprising VEGF and a TGFβ inhibitor (e.g., SB431542). In some aspects, the base culture medium comprises from about 10 ng/mL to about 100 ng/mL VEGF and from about 1 μM to about 20 μM of a TGFβ inhibitor (e.g., SB431542). In some aspects, the base culture medium comprises about 30 ng/mL VEGF and about 10 μM of a TGFβ inhibitor (e.g., SB431542).

In some aspects, a differentiation method provided herein comprises culturing cells in a base culture medium comprising TGFβ3 and a PDGF (e.g., PDGF-BB). In some aspects, the base culture medium comprises from about 1 ng/mL to about 5 ng/mL TGFβ3 and from about 2 ng/mL to about 8 ng/mL PDGF (e.g., PDGF-BB). In some aspects, the base culture medium comprises about 2 ng/mL TGFβ3 and about 4 ng/mL PDGF (e.g., PDGF-BB).

In some aspects, a differentiation method provided herein comprises:
(i) culturing pluripotent stem cells (e.g., iPSC) in a base culture medium comprising BMP4, VEGF, a GSK3 inhibitor, activin A, and a ROCK inhibitor;
(ii) culturing the cells of (i) in a base culture medium comprising VEGF and a TGFβ inhibitor; and
(iii) culturing the cells of (ii) in a base culture medium comprising TGFβ3 and a platelet-derived growth factor (PDGF) to form stromal cells or pericytes.

In some aspects, the method further comprises culturing the cells of (ii) in a base culture medium before (iii). In some aspects, the method further comprises (iv) culturing the cells of (iii) in a base culture medium. In some aspects, the cells of (ii) are not passaged before (iii).

In some aspects, a differentiation method provided herein comprises culturing PSC (e.g., iPSC) in a base culture medium comprising BMP4, VEGF, a GSK3 inhibitor (e.g., CHIR99021), and activin A. In some aspects, the base culture medium comprises from about 10 ng/mL to about 50 ng/mL BMP4, from about 10 ng/mL to about 100 ng/mL VEGF, from about 0.5 μM to about 3 μM of a GSK3 inhibitor (e.g., CHIR99021), and from about 5 ng/mL to about 50 ng/mL activin A. In some aspects, the base culture medium comprises about 30 ng/mL BMP4, about 50 ng/mL VEGF, about 1.5 μM of a GSK3 inhibitor (e.g., CHIR99021), and about 25 ng/mL activin A.

In some aspects, a differentiation method provided herein comprises:
(i) culturing PSC (e.g., iPSC) in a base culture medium comprising BMP4, VEGF, a GSK3 inhibitor (e.g., CHIR99021), and activin A;
(ii) culturing the cells of (i) in a base culture medium comprising VEGF and a TGFβ inhibitor (e.g., SB431542);
(iii) culturing the cells of (ii) in a base culture medium; and
(iv) culturing the cells of (iii) in a base culture medium comprising TGFβ3 and a PDGF (e.g., PDGF-BB) to form stromal cells or pericytes.

In some aspects, the method further comprises (v) culturing the cells of (iv) in a base culture medium. In some aspects, the cells of (ii) are not passaged before (iii).

In some aspects of any of the methods disclosed herein, the PSC (e.g., iPSC) are not fully confluent at the start of the culturing of (i). In some aspects of any of the methods disclosed herein, the PSC are at from about 50% to about 80% confluency at the start of the culturing of (i). In some aspects of any of the methods disclosed herein, the PSC are at about 50%, about 60%, about 70%, or about 80% confluency at the start of the culturing of (i).

In some aspects of any of the methods disclosed herein, the stromal cells are pericytes.

### III. Other Aspects

The present disclosure is also related to stromal cells made by any of the differentiation methods disclosed herein. The present disclosure is also related to pericytes made by any of the differentiation methods disclosed herein.

The present disclosure is also related to an organoid comprising stromal cells and/or pericytes disclosed herein. As used herein, the term “organoid” refers to a differentiated or partially differentiated, three dimensional (3D) cellular organism derived from pluripotent stem cells (e.g., iPSC) which is self-organized by densely accumulating cells in a controlled space. Such organisms can be crafted to replicate much of the complexity of an organ, or to express selected aspects of it, for example, only producing certain types of cells.

Methods for maintaining differentiated stromal cells, pericytes and organoids are well known and include culturing the cells or organoids in a cell culture medium described herein and/or cryopreservation. Methods for making an organoid typically include culturing cells in a 3D matrix. Suitable 3D matrices include, but are not limited to, polymers (natural or synthetic), ceramics, or composites. A 3D matrix can be in the form of: a hydrogel, a porous 3D scaffold, a rapid-prototyping scaffold, a foam, a sponge, a mesh, microparticles, fiber-like networks, mixtures of naturally occurring cell line-produced matrices such as Matrigel™, and combinations thereof, for example, microparticle-loaded hydrogels.

The present disclosure is also related to a method of promoting neovascularization or vascular development comprising administration of stromal cells, pericytes, or an organoid made by any of the differentiation methods disclosed herein.

The present disclosure is also related to a method of treating vasculitis or vasculopathy comprising administration of stromal cells, pericytes or an organoid made by any of the differentiation methods disclosed herein.

The present disclosure is also related to a method of treating a cardiovascular disease comprising administration of stromal cells, pericytes, or an organoid made by any of the differentiation methods disclosed herein. In some aspects, the cardiovascular disease is coronary artery disease (CAD), heart arrhythmias, heart failure, heart valve disease, pericardial disease, cardiomyopathy (heart muscle disease) or congenital heart disease.

The present disclosure is also related to certain differentiation media.

In some aspects, the present disclosure provides a differentiation medium comprising a base culture medium, BMP4, VEGF, a GSK3 inhibitor (e.g., CHIR99021), activin A, and a ROCK inhibitor (e.g., Y-27632). In some aspects, the differentiation medium comprises a base culture medium, from about 10 ng/mL to about 50 ng/mL BMP4, from about 10 ng/mL to about 100 ng/mL VEGF, from about 0.5 μM to about 3 μM of a GSK3 inhibitor (e.g., CHIR99021), from about 5 ng/mL to about 50 ng/mL activin A, and from about 1 μM to about 20 μM of a ROCK inhibitor (e.g., Y-27632). In some aspects, the differentiation medium comprises a base culture medium, about 30 ng/mL BMP4, about 50 ng/mL VEGF, about 1.5 μM of a GSK3 inhibitor (e.g., CHIR99021), about 25 ng/mL activin A, and about 10 μM of a ROCK inhibitor (e.g., Y-27632).

In some aspects, the present disclosure provides a differentiation medium comprising a base culture medium, VEGF and a TGFβ inhibitor (e.g., SB431542). In some aspects, the differentiation medium comprises a base culture medium, from about 10 ng/mL to about 100 ng/mL VEGF, and from about 1 μM to about 20 μM of a TGFβ inhibitor (e.g., SB431542). In some aspects, the differentiation medium comprises a base culture medium, about 30 ng/mL VEGF, and about 10 μM of a TGFβ inhibitor (e.g., SB431542).

In some aspects, the present disclosure provides a differentiation medium comprising a base culture medium, TGFβ3 and a PDGF (e.g., PDGF-BB). In some aspects, the differentiation medium comprises a base culture medium, from about 1 ng/mL to about 5 ng/mL TGFβ3, and from about 2 ng/mL to about 8 ng/mL PDGF (e.g., PDGF-BB). In some aspects, the differentiation medium comprises a base culture medium, about 2 ng/mL TGFβ3, and about 4 ng/mL PDGF (e.g., PDGF-BB).

In some aspects, the present disclosure provides a differentiation medium comprising a base culture medium, BMP4, VEGF, a GSK3 inhibitor (e.g., CHIR99021), and activin A. In some aspects, the differentiation medium comprises a base culture medium, from about 10 ng/mL to about 50 ng/mL BMP4, from about 10 ng/mL to about 100 ng/mL VEGF, from about 0.5 μM to about 3 μM of a GSK3 inhibitor (e.g., CHIR99021), and from about 5 ng/mL to about 50 ng/mL activin A. In some aspects, the differentiation medium comprises a base culture medium, about 30 ng/mL BMP4, about 50 ng/mL VEGF, about 1.5 μM of a GSK3 inhibitor (e.g., CHIR99021), and about 25 ng/mL activin A.

### EXAMPLES

Reference is now made to the following example, which together with the above descriptions illustrate some embodiments of the invention in a non-limiting fashion.

### Example 1

### Differentiation of Induced Pluripotent Stem Cells into Stromal Cells

An experiment was performed to differentiate stromal cells from human induced pluripotent stem cells (iPSC) (BJRiPS iPSC provided by Jintang Du) using the following protocol.

Day −1: The day before the beginning of the experiment, Matrigel® Growth Factor Reduced (GFR) Basement Membrane Matrix (Corning®, Product No. 354230) was placed on ice to melt overnight.

Day 0: On the day of initial iPSC seeding, 120 μL of Matrigel® GFR was added to 12 mL of Dulbecco's Modified Eagle Medium (DMEM)/F12 media (Thermo Fisher, Product No. 11320033) and mixed. 2 mL of this mixture was added to each well of a six-well plate and incubated for at least 1 hour at room temperature (RT).

Cell culture plates with iPSC at approximately 70% confluency were used for the initial seeding. Media was removed from the plates, and the iPSC were washed once with phosphate buffered saline (PBS) (Gibco, Product No. 12604-013). Next, 1.5 mL Accutase® (Biolegend, Product No. 423201) was added to the plates and incubated for 7 minutes at 37° C. The cells were then solubilized in Accutase® and passed through a strainer to collect cell clumps, and introduced into mTESR™ media (STEMCELL™ Technologies Inc.) to quench the Accutase®. This solution of cells was then counted using a MoxiCyte Viability Kit and Moxi Flow Instrument (ORFLO®).

Cells were then seeded at 200,000 cells per well in warm mTEST™ media with 10 μM Y-27632 (STEMCELL™ Technologies Inc.) and incubated overnight.

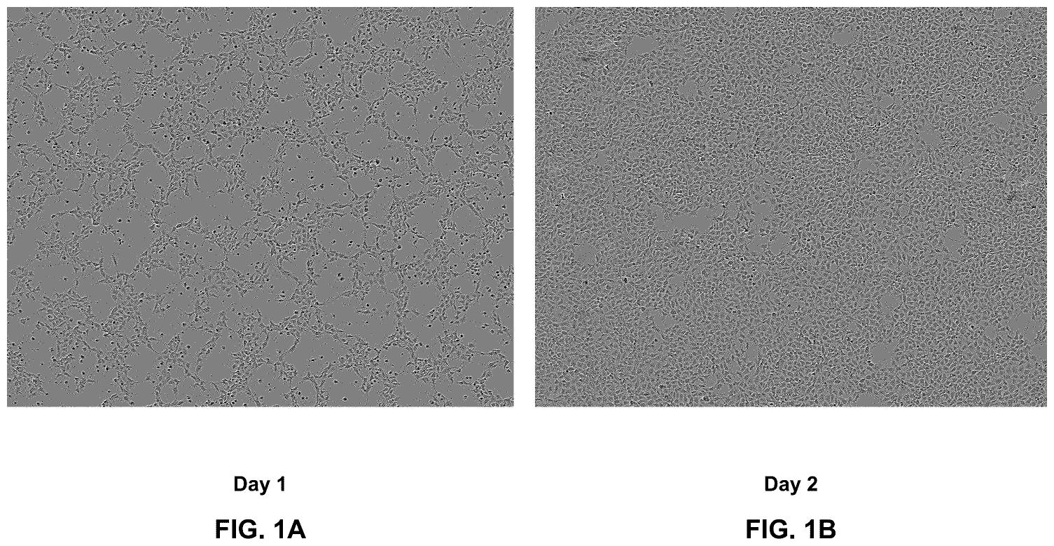

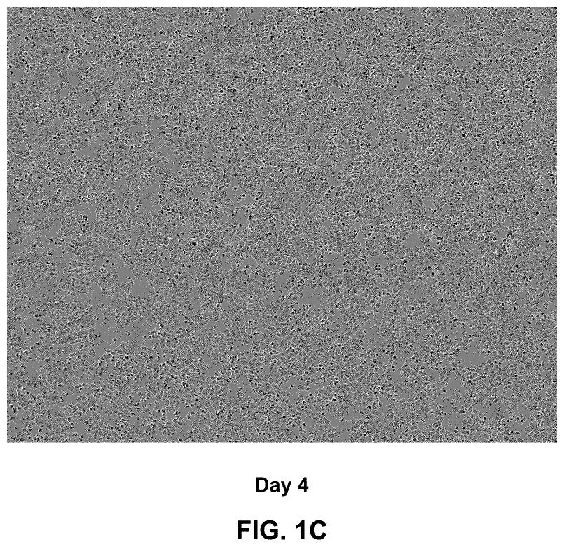

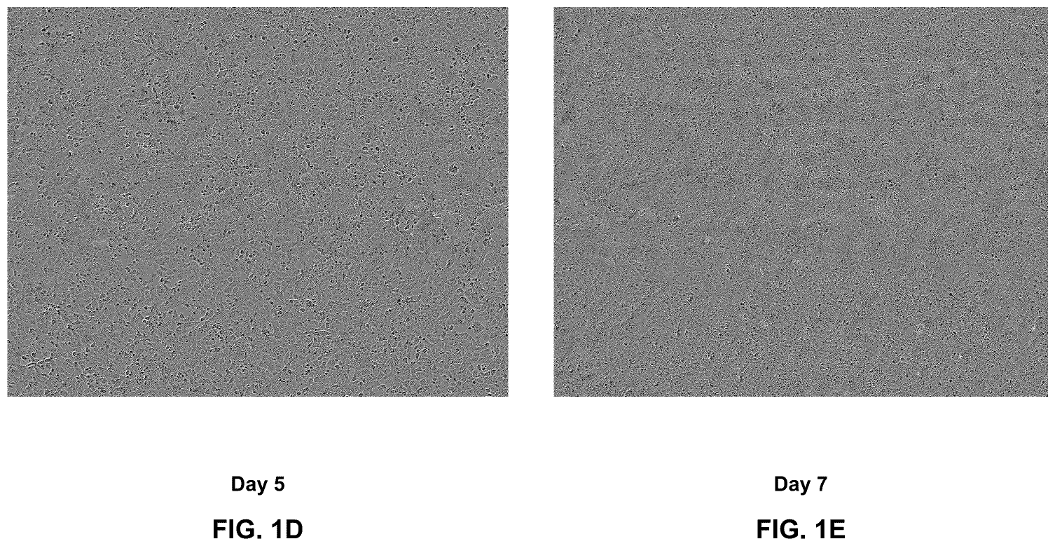

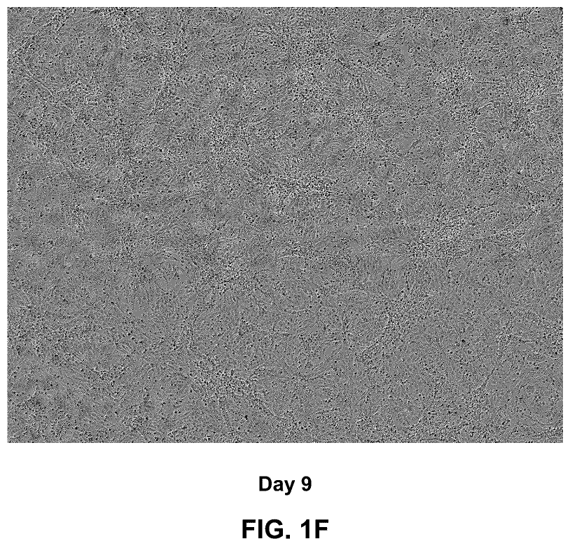

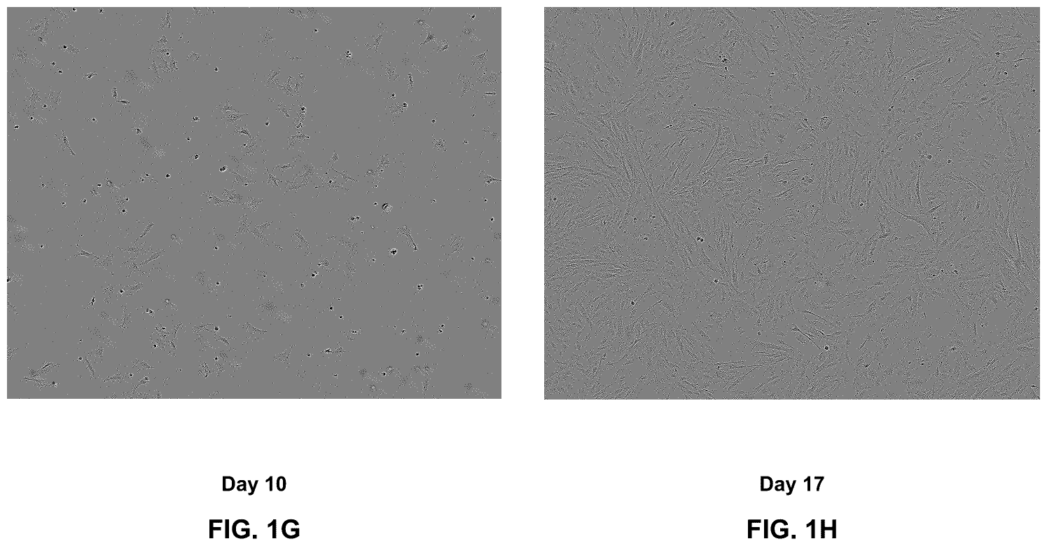

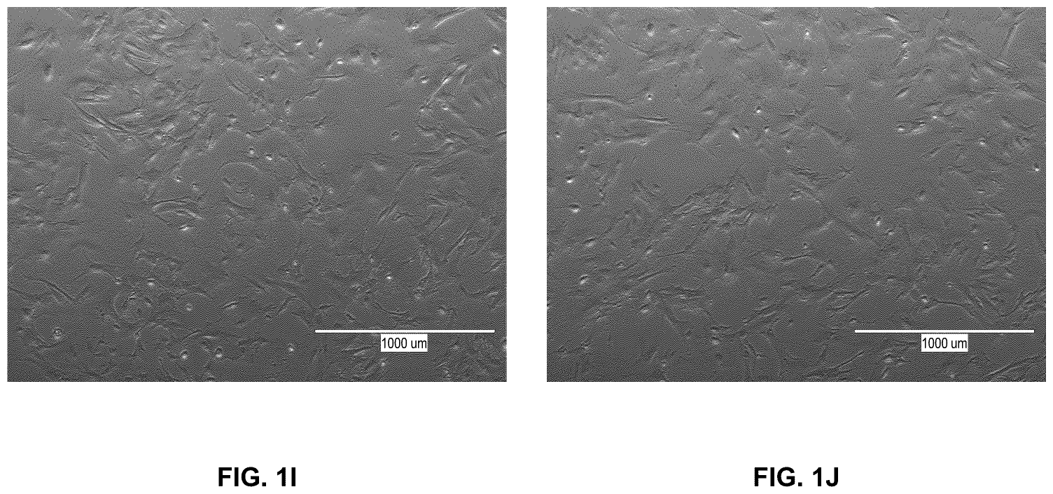

Day 1: Media was removed from culture plates with any unattached iPSC, and 2 mL of fresh, warmed mTESR™ with 10 μM Y-27632 was added per well to the cells. The cells were whole-well imaged using the Incucyte® Live-Cell Analysis System (Sartorius). An exemplary image is shown in FIG. 1A.

Day 2: Media was removed from culture plates with any unattached iPSC, and 2 mL of fresh, warmed mTESR™ with 10 μM Y-27632 was added per well to the cells. The cells were whole-well imaged using the Incucyte® Live-Cell Analysis System. An exemplary image is shown in FIG. 1B.

Day 3: Mesodermal differentiation. Media was removed from mostly confluent culture plates and replaced with 2 mL per well of filtered mesoderm induction media containing APEL™2 media (STEMCELL™ Technologies Inc.) with 25 ng/mL activin A (Peprotech, Product No. AF-120-14E), 30 ng/mL bone morphogenetic protein 4 (BMP4) (Peprotech AF-120-05ET), 50 ng/mL vascular endothelial growth factor (VEGF), 1.5 μM CHIR99021 (Biovision, Product No. 1748-5), and 10 μM Y-27632. Cells were whole-well imaged using the Incucyte® Live-Cell Analysis System and incubated for approximately 4 days. An exemplary image from day 4 is shown in FIG. 1C, and an exemplary image from day 5 is shown in FIG. 1D.

Day 7: Vascular differentiation. Media was removed from culture plates and replaced with 2 mL per well of filtered vascular induction media containing APEL™2 media with 50 ng/mL VEGF and 10 μM SB431542 (Tocris, Product No. 3211). Cells were whole well-imaged using the Incucyte® Live-Cell Analysis System and incubated for approximately 4 days with one replacement of vascular differentiation media. Exemplary images from day 7, day 9 and day 10 are shown in FIG. 1E, FIG. 1F, and FIG. 1G, respectively.

Day 11: Cell expansion. Cells were harvested by removing media, washed once with PBS, lifted by adding 1.5 mL Accutase®, and incubated for 7 minutes at 37° C. Accutase® was quenched with APEL™2 media and passed through a strainer to remove cell clumps. Cells were then counted using a Moxi Flow Instrument (ORFLO®), and plated on gelatin-coated T-75 flasks (Sigma, Product No. G1394; Falcon, Product No. 353136) at 800,000 cells per plate in 15 mL of complete EGM™-2 media (EGM™-2 Endothelial Cell Growth Medium-2 BulletKit™, Lonza, Product Nos. CC-3156 & CC-4176). Cells were allowed to grow to about 90% confluence, replacing media every 3 days with fresh complete EGM™-2 media. An exemplary image from day 17 is shown in FIG. 111.

Stromal/pericyte differentiation: Differentiation was started by removing the EGM™-2 media and adding 15 mL of Pericyte Growth Media (PromoCell) with 2 ng/mL transforming growth factor-beta3 (TGF-beta3 or TGFβ3) (Peprotech, Product No. 100-36E) and 4 ng/mL platelet derived growth factor-BB (PDGF-BB) (Peprotech, Product No. 100-14B). Cells were incubated for 3 days. Exemplary images of the resulting test differentiated cells are shown in FIG. 1I and FIG. 1J.

Next, test differentiated cells were validated by determining the expression level of various stromal and control markers using flow cytometry. A summary of the results from this validation showing relative expression levels is shown in Table 1. Results for test differentiated cells are shown, along with results from control endothelial cells, pericytes, fibroblasts and smooth muscle cells.

TABLE 1
Flow Cytometry Quality Control Panel

| | | | | | Test |
|---|---|---|---|---|---|
| | | | | Smooth | differentiated |
| | Endothelial | Pericyte | Fibroblast | Muscle | cells |
| CD31 | +++ | − | − | − | − |
| CD73 | + | +++ | +++ | +++ | +++ |
| CD90 | − | +++ | +++ | +++ | ++ |
| CD140a | + | ++ | ++ | + | ++ |
| CD140b | − | + | + | ++ | ++ |
| Desmin | + | ++ | ++ | ++ | ++ |

Next, test differentiated cells were further characterized with a tube formation assay using the following protocol. Test differentiated cells were co-cultured with human pulmonary artery endothelial cells (HPAEC) with or without 1 μM imatinib mesylate to inhibit angiogenesis, and tube formation was observed. Specifically, the wells of 96-well plates (Costar, Product No. 387) were coated with 30 μL of Matrigel® GFR. Plates were spun for 1 minute at 1,500 rotations per minute (RPM) and incubated for 30 minutes at 30° C. Cells were then lifted from culture plates, counted and resuspended in DMEM/F12 media such that 30 μL of media was available for each well. Each well received 30,000 HPAEC and 5,000 differentiated cells.

Green labeling of HPAEC was performed using Nuclight Green Lentivirus, Essen/Sartorius #4475. 100 μL of transfection reagent was added to 5 mL of media and mixed. In another 5 mL of media, 10 μL of needed lentivirus was added and mixed. Both 5 mL preparations were then added together, mixed, and added to an approximately 70% confluent plate of HPAEC or pericytes. Plates were allowed grow overnight in incubator.

Media and lentivirus preparation was removed and replaced with fresh media, and plates were incubated overnight. Plates were incubated for one week, using 1 μg of puromycin in the media for daily media changes

Differentiated cells were stained in 0.35 μM of Nuclight Rapid Red (Essen Bioscience) in PBS for 20 minutes at 37° C. The dye was then quenched with media and cells were distributed. For the tube formation assay, after gelling, 30 μL of cell suspension was carefully placed on top. Plates were loaded into the Incucyte® Live-Cell Analysis System and images were taken every hour.

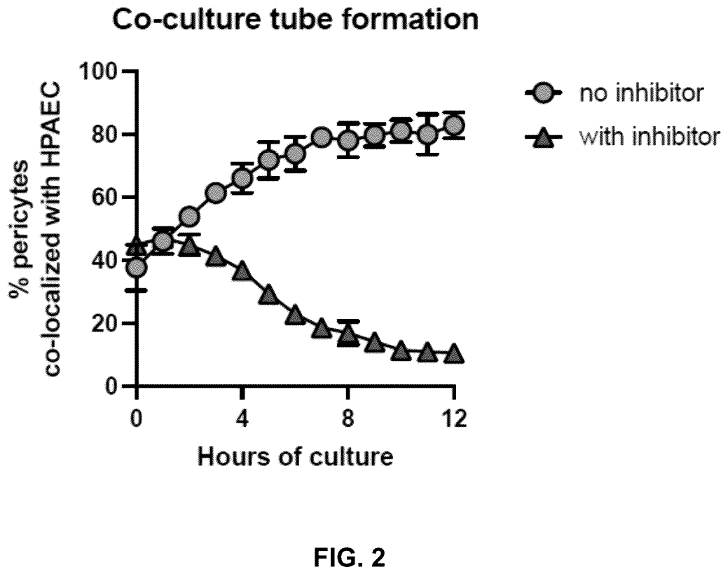

As shown in FIG. 2, test differentiated cells developed tube formation and co-localized with HPAEC, while control cells treated with imatinib mesylate did not.

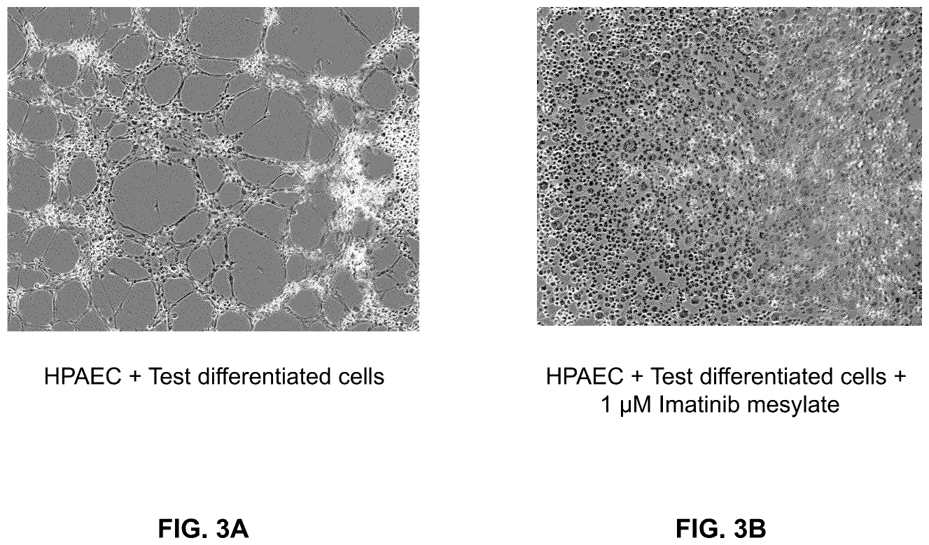

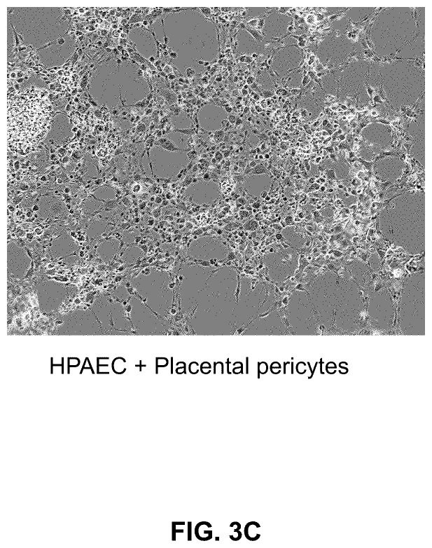

FIG. 3A-3C shows exemplary images from the three-hour timepoint for tube formation of co-cultures of HPAEC and test differentiated cells (FIG. 3A), HPAEC and test differentiated cells treated with imatinib mesylate (FIG. 3B), and HPAEC with placental pericytes (FIG. 3C). These results show that test differentiated cells associated with HPAEC tubes in a manner similar to primary pericytes.

Moreover, based on flow cytometry results and duration of tube formation stability and structure, test differentiated cells that grew the longest in expansion media had the best characteristics.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for differentiating pluripotent stem cells into stromal cells, comprising:
(i) culturing pluripotent stem cells in a base culture medium comprising bone morphogenetic protein 4 (BMP4), vascular endothelial growth factor (VEGF), a glycogen synthase kinase 3 (GSK3) inhibitor, activin A, and a Rho-associated, coiled-coil containing protein kinase (ROCK) inhibitor;
(ii) culturing the cells of (i) in a base culture medium comprising VEGF and a transforming growth factor β (TGFβ) inhibitor; and
(iii) culturing the cells of (ii) in a base culture medium comprising TGFβ3 and a platelet-derived growth factor (PDGF) to form stromal cells.

2. The method of claim 1, further comprising culturing the cells of (ii) in a base culture medium before (iii).

3. The method of claim 1, further comprising:
(iv) culturing the cells of (iii) in a base culture medium.

4. The method of claim 1, wherein the cells of (ii) are not passaged before (iii).

5. A method for differentiating pluripotent stem cells into stromal cells, comprising:
(i) culturing pluripotent stem cells in a base culture medium comprising BMP4, VEGF, a GSK3 inhibitor, and activin A;
(ii) culturing the cells of (i) in a base culture medium comprising VEGF and a TGFβ inhibitor;
(iii) culturing the cells of (ii) in a base culture medium; and
(iv) culturing the cells of (iii) in a base culture medium comprising TGFβ3 and a PDGF to form stromal cells.

6. The method of claim 5, further comprising:
(v) culturing the cells of (iv) in a base culture medium.

7. The method of claim 5, wherein the cells of (ii) are not passaged before (iii).

8. The method of claim 5, wherein the culture medium of (i) further comprises a ROCK inhibitor.

9. The method of claim 1, wherein the ROCK inhibitor is Y-27632.

10. The method of claim 9, wherein Y-27632 is present in the culture medium at a concentration of about 10 μM.

11. The method of claim 1, wherein the GSK3 inhibitor is CHIR99021.

12. The method of claim 11, wherein CHIR99021 is present in the culture medium at a concentration of about 1.5 μM.

13. The method of claim 1, wherein BMP4 is present in the culture medium at a concentration of about 30 ng/mL.

14. The method of claim 1, wherein VEGF is present in the culture medium at a concentration of about 50 ng/mL.

15. The method of claim 1, wherein activin A is present in the culture medium at a concentration of about 25 ng/mL.

16. The method of claim 1, wherein the TGFβ inhibitor is SB431542.

17. The method of claim 16, wherein SB431542 is present in the culture medium at a concentration of about 10 μM.

18. The method of claim 1, wherein TGFβ3 is present in the culture medium at a concentration of about 2 ng/mL.

19. The method of claim 1, wherein the PDGF is PDGF-BB.

20. The method of claim 19, wherein PDGF-BB is present in the culture medium at a concentration of about 4 ng/mL.

21. The method of claim 1, wherein the stromal cells are pericytes.

22. The method of claim 1, wherein the pluripotent stem cells are not fully confluent at the start of the culturing of (i).

23-24. (canceled)

25. Stromal cells made by the method of claim 1.

26. Pericytes made by the method of claim 1.

27. An organoid comprising the stromal cells of claim 25.

28. An organoid comprising the pericytes of claim 26.

29. A differentiation medium comprising a base culture medium, BMP4, VEGF, a GSK3 inhibitor, activin A, and a ROCK inhibitor.

30. (canceled)
